(19) United States
(12) Patent Application Publication
(10) Pub. No.: US 2013/0022663 A1
(43) Pub. Date: Jan. 24, 2013

(54) Method of making and using a topical medicated formula and delivery system to use to prevent and treat breast diseases and breast cancer as well as use as skin wellness and anti-wrinkle formula

(71) Applicant: Kevin Buckman, Stockton, CA (US)

(72) Inventor: Kevin Buckman, Stockton, CA (US)

(73) Assignee: VARS INC

(21) Appl. No.: 13/135,920

(22) Filed: Jul. 19, 2011

(51) Int. Cl. A61K 9/127; A61K 9/50; A61P 35/00; A61P 29/00; A61P 37/00; A61K 35/60; A61P 3/02; A61P 3/10; A61P 9/00; A61K 35/56; A61P 15/00

(52) U.S. Cl. 424/450; 424/547; 424/490; 424/523

(57) ABSTRACT

Breast cancer and breast disease are common in women and becoming more frequent in men. Early treatment is vital for better outcomes. This formula consists of a liquid anti-disease topical and/or intraductal anti-breast-disease and anti-cancer—including against precursors to cancer and atypical cells—formula that can be delivered to the source where breast cancer begins by use of a number of bio-available components and multiple diffusion and delivery systems, preventing and/or treating maladies that include, but are not limited to degenerative diseases and accumulation of toxins in the breast tissue.

May include homeopathic remedies to be used topically or inside the breast with a delivery method to those regions and may be processed with or without photonic programming or an ionic delivery system for greater effectiveness.

### BACKGROUND OF THE INVENTION

This is a method of treating and preventing breast cancer in men, women or animals, with a formula and a means of delivery of the formula with topical use or with a breast patch or needle injection of the formula into the breast. It is a method of making and using a topical medicated formula and delivery system to use to prevent and treat breast diseases and breast cancer.

This formula consists of a liquid anti disease anti cancer formula that can be delivered to the source where breast cancer begins, which is the ductal and topical system of the breast, with an topical infusion or via a suction and pumping action of the device to irrigate the breast tissue, or a topic means of delivery with the anti breast cancer anti breast inflammation anti breast cell atypia and hyperplasia and breast wellness formula.

This formula may use chemotherapy agents, anti-inflammatory agents, with or without an ionic delivery systems for more reliable absorption into the breast with a topical agent or through the use of a device that is topical application, patch, any acceptable form of use, or via a suction and pumping action of the device to irrigate the breast tissue with the anti breast cancer anti breast inflammation contained in this invention that can also treat or prevent breast cell atypia, cancer, and hyperplasia as well as promote breast wellness.

This formula may include photonically programmable water and neutraceuticals in a form that has medicinal activities that may include but not limited to: decrease in oxidative stress to cells, support of healthy DNA, RNA, mitochondria with its anti-inflammatory, anti cancer, anti angiogenesis, and anti-aggregatory actions of stopping clots, by use of an alkaline charge of the water and minerals, preventing a buildup of elements responsible cancer or the precursors of cancer, or atypia or inflammatory tissue or cancerous cells.

This formula may block the adverse effect on synthesis of prostaglandins which are chemicals that play a part in inflammation and harmful thrombotic activities, acidosis, and adverse environmental terrain of the breast and surrounding tissue, and thereby becoming a breast wellness formula by treating or prevention of mutations of breast cells and tissues, and remediation of carcinogens, and restoring the homeostasis of the cells and tissues of the breast.

This is a method of making a topical breast wellness formula with delivery systems to allow for deep penetration inside the breast when applied to the nipple and breast region to treat and prevent breast cancer and breast disease.

The formula contains anti-oxidant, anti-inflammatory, chelating and other agents to combat disease and breast cancer. One of the ingredients has a water soluble form of Fucoidan which has effects the aging process that is caused in part by degeneration of cells and surrounding tissues.

The etiology of breast cancer has been linked to lack of nutrients and metabolic deficiencies, toxins in food and the environment, and cellular environmental terrain, all which is treated by this formula, including the treatment.

The inventor of this formula has over 3 decades with extensive research and clinical experience of 30 years, and has written books on this subject.

This formula may use of antioxidants with and without soluble forms of seaweed and fucoidan, minerals that create an environment that are harmful to cancer and its precursors to cancer, or causing agents and other antioxidants that have anti-oxidant, anti-inflammatory, pH stabilizing, pain relieving effects and Nutraceutical benefits, beneficial effects against the aging process that is caused in part by inflammatory cells, damaged cells by oxidative stress, or degeneration of cells and/or the surrounding tissues.

This formula may include Delivery Systems with may contain the following: a programmable water, alkaline substances, use of nano-sized particles and ingredients, alkaline water, liposomal delivery, methods of making substances water and lipid soluble, making substances bioavailable, by reduction of inflammation which improves absorption, and other methods known, not known or yet to be discovered, and with or without trace minerals, antioxidants and nutrients that collectively decrease oxidative stress to cell with anti-inflammatory, and anti-aggregatory actions of stopping clots and build up of elements responsible for heart attack, stroke, and sudden cardiac arrest, blocks the adverse effect on synthesis of prostaglandins which are chemicals that play a part in inflammation and harmful thrombotic activities, the stable form and the selection of antioxidants in this formula inhibit inflammation that has been linked to chemicals or an acid state to the tissue or the accumulation of toxins in the tissue and with or without the use of nanoneutraceuticals. These nanoneutraceuticals are formed by use of this device wherein a nutrient can be made more useful for the body such as a delivery system for the nutrients as part of metabolic pathways and entrance into cells.

The photonic and ultraviolet processing of the formula may produce nano-sized nutraceutical particulate materials for greater bioavailability and usefulness for a breast wellness and anti breast disease formula. These nutrients are also photoceutical, with photonic energy processing the water or other nutrient for medicinal use with or without the use of breast nipple catheter, breast ductal, infusion and irrigation with a pump or use of a hand held device.

This is a means of breast infusion of the formula that may be done with breast ductal medicated infusion and irrigation with a pump or use of a hand held device or mechanical means of flow warm water around the nipple area of the breast and allow for flow into the nipple and inside of the breast ductal system and breast tissue, with or without a cup that allows for such a flow, with or without the assistance of a breast nipple topical and topical catheter.

This is a medicated formula for breast diseases of all kinds or for prevention of breast diseases of all kinds with or without the use of liposomes for enhanced use of the formula included in this patent, with or without a device to assist delivery to the region of the breast and breast ductal region via the breast nipple.

Medicated formula with or without the use of liposomes, with or without the following: alkaline salts and other nutrients, anti-inflammatory agents, fucoidan, with or with one or more of the following chemo therapy agents such as but not limited to: Abraxane (paclitaxel protein-bound particles for injectable suspension), Adriamycin (doxorubicin), Aredia (generic name, pamidronate disodium), Arimidex (anastrozole), Aromasin (exemestane), Chemotherapy Regimens of different kinds known and not known and yet to be discovered, Cytoxan (cyclophosphamide), Ellence (epirubicin), Evista (raloxifene), Fareston (toremifene), Femara (letrozole), Herceptin (trastuzumab), Megace (megestrol), Tamoxifen, Taxol (paclitaxel), Taxotere (docetaxel), Xeloda (capecitabine), Zoladex (goserelin acetate), Zometa (generic name, zoledronic acid), and other agents.

The elements contained in this formula for the treatment or prevention of breast diseases of all kinds, may be processed with or without photonic programming or an ionic delivery system for greater effectiveness and use against degenerative diseases or cancerous or precancerous diseases, precursors of breast disease, inflammatory breast disease, or accumulation of toxins in the breast tissue that tend to concentrate in the breast such as, but not limited to mercury, cadmium, lead, heavy metals, benzene, furans, organophosphates, petroleum derivates, and other toxic substances.

The elements contained in this formula for the treatment or prevention of breast diseases of all kinds may contain anti-inflammatory and reparative properties and as an anti-aging agent and wellness formula, with or without fucoidan for its benefits, and with or without alkaline minerals for homeostasis of breast tissue, with or without homeopathic formula for breast disease, hormonal balancing, and other nutrients.

Some examples of nutrients that can be used include: Omega Fish Oil Concentrate, Flax Seed Oil, Borage Oil. In addition, there may be a Mineral Bio-Matrix, Trace Minerals, MSM, Piper Nigrum as an ionic delivery system, and a Natural Antioxidant Complex that may consist of Rosemary Oil, Alpha Tocopherol, Astaxanthin, and Algal Oil. Algal Oil can be used as a stabilizing agent to prevent oxidation of the essential fatty acids that cause oxidative stress to cells when not stabilized.

Degenerative diseases such as breast cancer have been linked to a lack of nutrients as well as metabolic deficiencies and chemical imbalances, toxins in food, acidosis, and toxins from the environment. The combination and individual ingredients in this formula have particular benefits on breast disease, cancerous and precancerous breast lesions, and breast degenerative disease.

Components in this formula have the abilities to assist the cells of the breast against inflammation and disease by its assisting with assimilation of toxins, removing, eliminating, diminishing, processing, scavaging free radicals associated with cycles of cell disease, problems with cell and systemic metabolism, cellular acidosis, and the inflammatory process.

This may include the use of fucoidan with or without an ionic delivery system has been shown to provide benefits for diabetes, inflammatory processes, immune disease and heart disease. The brown seaweed with Fucoidan, beta glucans and mucopolysaccarides are key active nutrients to improve the health of the cells by many metabolic and nutritional mechanisms. These do so by their broad-spectrum anti-oxidant effect with minerals other nutrients in the fucoidan and brown seaweed in this formula. This is due in part due to the absorption and elimination of oxidants and free radicals such as the heavy metals and toxins that play an important role in the pathogenesis.

Use of a fucoidan for its Anti-aging and wellness formula in an ionic delivery system using alkaline minerals in an oxide state, trace minerals, herbal extracts, antioxidants and metabolic nutrients for cell health and the treatment of breast health

This formula has anti-inflammatory agents, plus ionic delivery systems for more reliable absorption through the use of a form that is topical infusion or via a suction and pumping action of the device to irrigate the breast tissue with the anti breast cancer anti breast inflammation anti breast cell atypia and hyperplasia.

The nutrients are sourced from the following due to their quality, bioavailability and level of essential fatty acids and the ability to be used with the stabilization process and with the ionic delivery process with this method of making an antioxidant health care product: Omega Fish Oil Concentrate, Flax Seed Oil, Borage Oil. In addition, there is an Ionic trace Minerals, MSM, Piper Nigrum as an ionic delivery system, and a Natural Antioxidant Complex that consists of Rosemary Oil, Alpha Tocopherol, Astaxanthin, and Algal Oil. Algal Oil is used as a stabilizing agent to prevent oxidation of the essential fatty acids.

These remedies may be selected for treating various conditions of the breast, especially breast cancer: Bufo vulgaris, Conium maculatum, Graphites, Mercurius, and Silicea.

The use of these and other homeopathic ingredients can be selected based on history of exposure to toxins and with clinical findings and subjective complaints, as well as the condition of the patient. These are formed from heavily diluted preparations, and these may be used with effects similar to the disease's symptoms. This dilution is usually quite extensive, and often continues until no molecules of the original substance are likely to be found by routine testing.

One in five women who are breast feeding has elevated mercury levels in their breast milk and many women have concentrations of mercury and heavy metals in the breast. The treatment for those who are tested, exposed, possibly exposed to mercury and heavy a homeopathic formula may include Hepar sulph., Kali iod., Lachesis, Nitric acid, Phytolacca, and Silicea, and Mercurius would not be selected where there is a concern about mercury poisoning.

There are other ingredients that can be included for their properties, such as but not limited to: apis can be used when there is inflammation, atypia, or hyperplasia of breast tissue. Arsenicum can be used if there is lowered immunity or breast cancer, or if nipples are retracted. Asterias can be used with swelling of breasts or with any stage of breast cancer, even in ulcerative stages. Baric iodide can be used with cancer of breasts, suspected autoimmune problem that is linked to the breast cancer or need for iodide. Belladonna, or Deadly Nightshade for breast inflammation and cancer. Bromium for swelling of breast tissue. Calcareas for precancerous conditions and local pain. Cancer nosode or carcinosinum for breast cancer. Chimaphila or pipsissewa for breast tumors.

Conium for breast cancer and inflammation Hepar sulphuris calcareum for breast cancer and for remediation of heavy and other metals. Cundurango for breast wellness, Hydrastis for immune support for breast cancer and precancerous conditions, Nitric acid for breast wellness, Phosphorus for cancer of breast, sorinum for breast inflammation, kalis for balance, lycopodium, Silicea for inflammation and infection, and/or thuja for breast wellness.

This formula and method of its delivery to abnormal breast tissue or to prevent the formation of cancer or precursors to cancer or atypia or other form of abnormal breast tissue.

This formula consists of a liquid anti disease anti cancer formula that can be delivered to the source where breast cancer begins in the breast.

This is an topical and/or interductal infusion or via a suction and pumping action of the device to irrigate the breast tissue with the anti breast cancer anti breast inflammation anti breast cell atypia and hyperplasia.

This formula has anti-inflammatory agents, plus ionic delivery systems for more reliable absorption through the use of a form that is topical infusion or via a suction and pumping action of the device to irrigate the breast tissue with the anti breast cancer anti breast inflammation anti breast cell atypia and hyperplasia and breast wellness.

Method of making an alkaline product with its anti-inflammatory and antioxidant formula with nutrients wherein nutrients can be used and maintained in a suitable form to prevent the toxic effects of oxidation of product and free radical activity in a formula for immune support, antioxidant activity, anti coagulant activity and other activities, as a wellness formula. The use nutrients in this formula that can be bioavailable and have anti-oxidant activity that may be broad spectrum, work as metabolic catalysts for the body, create better oxygen delivery and nutrient delivery to cells due to the metabolic, nutrient, and electric affect on the body that has wellness properties, anti-aging, immune enhancing and disease resistant activities, protective benefits for breast tissue.

Problems that occur with arthritis, asthma, pain syndromes, and chronic diseases are inflammation and cell injury. The formula is designed to deliver maximum benefits for these conditions. The senior author has done a lot of research in the area of chronic and degenerative diseases during the last 30 years. The inventor of this formula has used a form of this invention with a blend of some of the following substances in his formula as well as others in clinical trials in women with good results and no adverse reactions reported: Purified Alkaline Water, alcohol, Glycerine, Flax Seed Complex, Safflower Oil, Fucoidan Native Extract, Brazil Nut Oil, Hazelnut Oil, MSM, Liposome formula, Green Lipped Mussel Extract, Natural Fragrance, Natural Preservative Complex. These were used in ratios that allowed for best texture and consistency. Use of ratios were tested extensively for absorption, thickness, easy of use, use in a tube, use in containers of all kinds, design for preferred texture and feeling of skin after use without oily or adverse feeling after use, best acceptance by users, use of application, feeling of the skin after use, and other factors for marketability of this product.

There is concern that mammograms can damage breast cells with moderate to severe compression and radiation, so much is the concern that medical scientists have suggested getting no more than 10 mammograms per lifetime of a women.

There is also legitimate concern of women and scientists that mammograms may also spread breast cancer to other parts of the body due to the breast tissue being crushed in the process of doing the mammogram.

Breast cancer is a common cancer in women and cases in men have been increasing.

Early diagnosis and treatments are vital for better outcomes.

Each year approximately 200,000 new patients with breast cancer are diagnosed in North America, and 50,000 of those will die of the disease. Despite billions of dollars poured into research, public awareness, and improved treatment, the statistics have shown only modest improvement over the past twenty years. The slight increase in survival is due to earlier diagnosis but mostly to improved treatment.

For breast cancer prevention with its Non-Invasive or invasive use of infusion of formula for the treatment or prevention of breast disease, this can be done in a non invasive or invasive manner with or without a breast nipple catheter and with or without a hand held device or with or without an electrical or non electrical pump and suction device for use with the medicated solution for the breast disease or breast disease prevention. This formula with a device or method of delivery can be used with a Breast Wellness and Screening Program.

If a woman has a positive breast test for atypical cells, cancer situ, cancerous or precancerous cells in the breast or breast ductal region, there are many actions that women can take to be proactive. Atypical cells may be the next step to breast cancer, just like those of the cervical Pap test for cells that are not normal, so intervention with the device and formula of this invention can treat or prevent disease.

In women who have no fluid return or cells on NAF testing (Nipple Aspirate Fluid), this may be due to blockage by cancerous, inflammatory tissue, atypia, hyperplasia, fibrous tissue, scaring, and other abnormal breast and cellular tissue, these conditions may benefit from the formula and delivery systems of this invention.

There is clear evidence that breast cancer has subgroups based on age. In younger patients the disease is more aggressive and difficult to treat. The mortality in this group is significantly higher than in the older age group. Early diagnosis is still the watchword for improving survival, but unfortunately early diagnosis is most difficult in the under 50-age group. The reasons for this difficulty are varied, but the major problems have been that younger women are concerned about cancer, but frequently in denial. They have a poor record of compliance for routine annual screening and doctor's physical exam and what to do about the cancer if it is found.

Younger women tend to have denser tissue because their bodies are still producing lots of hormones which stimulate breast tissue growth. This formula can include a means of hormonal balancing that is needed for breast wellness, prevention and treatment of breast disease and cancer.

Mammograms, the “gold standard” of breast cancer diagnosis are only 50% sensitive to cancer in this dense breast group. Even with newer digital mammography, which is present in a few diagnostic centers, the sensitivity only reaches to 70%. This means that one third to one half of all women, especially younger ones, will fail to have their breast cancer diagnosed early using traditional approaches. This is likely a major reason why death rates from breast cancer have changed very little over the past decades, and why there is a need for early intervention and treatment.

Over these two decades many new approaches to early diagnosis have been tried and abandoned. Light transmission diagnosis, electrical resistance, contact thermography has limitations. Breast self-exam is still the only recommended standard for women under the age of 40, though it sensitivity (ability to detect any disease) and specificity (accuracy in predicting cancer) are extremely low. It is still recommended by all the cancer societies and the federal government for two reasons. The first is that the incidence of cancer in the under-40 age group is low, and therefore one would have to spend a lot of insurers' money on other screening tests to pick up relatively few cancers. The second is that self-exam gives the impression that women can actually help themselves and be proactive against cancer, The actual results indicate it is a very poor method of screening, picking up less than 5% of all breast cancers as currently performed by most women. For women of this age group and for older women with dense breasts, there has been no solution to the problem of early diagnosis and treatment, so a formula and method of delivery of a medicated formula is needed for early intervention and prevention of breast diseases.

These are the millions of women who do not wish to get annual mammography for fear of the radiation and fear of chemotherapy, surgery, and radiation. In fact, a recent Medical Meeting of consultants and OB/GYN meeting at UCSF, have suggested that a woman get no more than 10 Mammograms in her lifetime.

A recent National Cancer Institute study confirmed an increased risk of breast cancer among women getting annual mammography. There is a large group of women concerned about this risk of mammography and risk of breast cancer, and a medicated formula is needed for early intervention and prevention of breast cancer, and since, based on many years of research by the author of this patent, there is a window of 8 to 10 years where breast cancer is “dormant,” with breast cells still mutating or still precancerous, and a progression to breast cancer and metastasis may be prevented on early intervention, based on many years of research by the author of this patent. In addition, there may be an opportunity to remediate local toxins in the breast that are carcinogenic.

1. This is a method of treating and preventing breast cancer and breast disease in men, women or animals, with a formula and a means of delivery of the formula that can be used as a topical, topical, ductal, topical infusion, injectable, subcutaneous, or other means of delivery that consists of a formula that creates repair, or cellular-environmental, pericellular homeostasis in the region of the breast and surrounding tissue, and is a means of providing early treatment that is vital for better outcomes in breast cancer and breast disease. This formula consists of a topical cream or liquid anti-disease for topical use on the nipple and breast area and chest where there are mammary cells with a formula and delivery systems that are anti breast disease and anti-cancer formula that can be delivered to the source where breast cancer begins by use of a number of bioavailable components and multiple diffusion and delivery systems. The preferred embodiment of the formula consists of a medicated and programmable water and Purified Alkaline Water, alcohol, Glycerine, Flax Seed Complex, Safflower Oil, Fucoidan Native Extract, Brazil Nut Oil, Hazelnut Oil, MSM, Liposome formula, Green Lipped Mussel Extract, Natural Fragrance, Natural Preservative Complex.
These are used in ratios that allowed for best texture and consistency. Use of ratios allow for absorption, pricing, thickness, ease of use, use in a tube, use in containers of all kinds, design for preferred texture and feeling of skin after use without oily or adverse feeling after use, best acceptance by users, use of application, feeling of the skin after use, and other factors for marketability of this product.
This formula and method of its delivery is to treat abnormal breast tissue or other region of the body to be treated, or to prevent the formation of cancer or precursors to cancer or atypia or other form of abnormal breast or other tissue before it spreads or becomes breast cancer and causes metastasis to the brain, liver, bone and other parts of the body.
The preferred embodiment of this formula and means to treat breast diseases all kinds may include fucoidan in an ionic or non ionic formula, with or without homeopathic remedies that may be used topically or inside the breast and nipple with a method of delivery to those regions, that can used in a water soluble form that is pH balanced and can be tolerated in the region of the breast and ductal system of the breast. This remedy may be homeopathic and may consist of a means to control hormonal levels and effects of hormonal imbalances. A homeopathic remedy and other nutrients may be used for improving the balance of pH, estrogen, progesterone, and testosterone, as well as its other benefits which may include anti-inflammatory homeopathic ingredients, anti-cancer homeopathic ingredients, and other homeopathic agents that can prevent or treat breast cancer. The formula may consist of one or various delivery systems, such as but not limited to, a liposomal or liposomal like delivery system, diffusion and dissemination vehicles, an instrument for infusion to tissue, and other means. The formula may contain a water soluble form of fucoidan, alkaline minerals, germanium sexiquioxide, MSM, rainbow broad spectrum antioxidants, citric acid, ascorbic acid, buffers, alkaline water, alkaline minerals, pH stabilizers, programmable and or structured water, hydrogen water, nano water, water that is wetter, water that is a delivery system, water in microclusters, water that donates positive hydrogen ions, or other substances that are known, not known or yet to be discovered. It is a method of making and using a topical medicated formula and delivery system to use to prevent and treat breast diseases and breast cancer. This formula may include propriety Delivery Systems and methods of use witch may contain the following: a programmable water, alkaline substances, use of nano-sized particles and ingredients, ozone, oxygen producing substances, alkaline water, liposomal delivery, methods of making substances water and lipid soluble, making substances bioavailable, by reduction of inflammation which improves absorption, fights against a hypoxic state or provides an alkaline and oxygen rich environment to the cells being treated in the breast and other regions of the body, and other methods known, not known or yet to be discovered, in conjunction with key ingredients and components of this patent. It is a method of making and using a topical medicated formula and delivery system to use to prevent and treat breast diseases and breast cancer as well as use as skin wellness and anti-wrinkle formula for any location on the body.

2. A method of making an anti-inflammatory and antioxidant formula with or without omega fatty acids in a stable form that decreases oxidative stress to cell with anti-inflammatory, and anti-aggregatory actions of stopping clots and build up of elements responsible for breast disease, with or without the following: astaxanthin, white willow bark, ascorbate, apple pectin, other antioxidants, to be given topically in small amounts, or other form, topical form or as a gel or with liposomal delivery, or sustained release microencapsulation, wherein part of the formula is can be processed with an ionic delivery system or other form of a delivery system or additives for bioavailability that are known, not yet known, or yet to be discovered for greater effectiveness of the formula for use with breast diseases of all kinds or with other benefits known or not yet known, as claimed in claim 1, and

3. A method of making a nutrient enhancing formula that may be beneficial to breast disease, such as fibrous breast disease, mastitis, benign and malignant tumors of the breast, and precancerous breast conditions, and for breast autoimmune syndromes by making the nutrients more bioavailable and antioxidants in a stable and useful form and may use a Delivery System to increase of the benefits of this formula, as claimed in claim 1, and

4. Method of making an alkaline product with its anti-inflammatory and antioxidant formula with nutrients wherein nutrients can be used and maintained in a suitable form to prevent the toxic effects of carcinogens and toxins with free radical activity in a formula for immune support, antioxidant activity, anti coagulant activity and other activities, as a breast wellness formula. The use nutrients in this formula that can be bioavailable and have anti-oxidant activity that may be broad spectrum, better oxygen delivery and nutrient delivery to cells due to the metabolic, nutrient, and electric affect on the body that has breast wellness properties, and other benefits, as claimed in claim 1,

5. This is a method and use of nutrients and alkaline water with antioxidant activity and with to combat inflammation and degeneration of breast cells and cell contents which are known to be part of the pathogenesis of breast disease, as claimed in claim 1, and

6. This patent may include the addition of the metabolic and nutrient rich delivery system, ionic delivery or other delivery system of one or more of the following: the colloidals, minerals, and other substances that supports, balances and enhances the full spectrum anti-oxidant and nutrients benefits for breast cell health, and those nutrients, and a method of making a bioavailable formula for treatment inside of the breast to prevent or to treat breast cancer, as claimed in claim 1, and

7. This is a method of treating breast cancer with a formula and a means of delivery of the formula with a topical application. This formula and method of its delivery is to treat abnormal breast tissue or to prevent the formation of cancer or precursors to cancer or atypia or other form of abnormal breast tissue.
This formula and means to treat breast diseases all kinds may include homeopathic remedies that may be used topically or inside the breast and nipple with a method of delivery to those regions, and may include other regions of the body such as skin, prostate, colorectal, and other sites of the body, as claimed in claim 1.

8. This is a method of making and using a formula that blocks the adverse effect on synthesis of prostaglandins which are chemicals that play a part in inflammation and harmful thrombotic activities, acidosis, and adverse environmental terrain of the breast and surrounding tissue, and thereby becoming a breast wellness formula by treating or prevention of mutations of breast cells and tissues, and remediation of carcinogens, and restoring the homeostasis of the cells and tissues of the breast as claimed in claim 1, and

9. This is a method of making a formula that treat breast disease with single dosing, intermittent, or continual treatment and consists of a breast wellness and cancer treatment, This is a method of treating and preventing breast cancer in men, women or animals, with a formula and a means of delivery of the formula with topical use or with a breast patch or needle injection of the formula into the breast, as claimed in claim 1, and

10. Provides a method of breast cell health by homeostasis, cell treatment, that may include a balance of herbal, mineral, and other substances associated with breast health, anti-inflammatory benefits for atypia and hyperplasia of the breast occur when the pathogenesis is inflammation and other pathological processes, which causes more mutations and leads to cancer, or cause breast disease, as claimed in claim 1, and

11. These homeopathic remedies may be selected for treating various conditions of the breast, especially breast cancer: Bufo vulgaris, Conium maculatum, Graphites, Mercurius, and Silicea. The use of these and other homeopathic ingredients can be selected based on history of exposure to toxins and with clinical findings and subjective complaints, as well as the condition of the patient and are made from serial dilutions that are diluted very many times.
One in five women who are breast feeding has elevated mercury levels in their breast milk and many women have concentrations of mercury and heavy metals in the breast.
The treatment for those who are tested, exposed, possibly exposed to mercury and heavy a homeopathic formula may include Hepar sulph., Kali iod., Lachesis, Nitric acid, Phytolacca, and Silicea, and Mercurius would not be selected where there is a concern about mercury poisoning.
Apis can be used when there is inflammation, atypia, or hyperplasia of breast tissue. Arsenicum can be used if there is lowered immunity or breast cancer, or if nipples are retracted. Asterias can be used with swelling of breasts or with any stage of breast cancer, even in ulcerative stages. Baric iodide can be used with cancer of breasts, suspected autoimmune problem that is linked to the breast cancer or need for iodide. Belladonna, or Deadly Nightshade for breast inflammation and cancer. Other components and their targets include, but are not limited to: Bromium for swelling of breast tissue, Calcareas for precancerous conditions and local pain, Cancer nosode or carcinosinum for breast cancer. Chimaphila or pipsissewa for breast tumors, Conium for breast cancer and inflammation, Hepar sulphuris calcareum for breast cancer and for remediation of heavy and other metals, Cundurango for breast wellness, Hydrastis for immune support for breast cancer and precancerous conditions, Nitric acid for breast wellness, Phosphorus for cancer of breast, sorinum for breast inflammation, kalis for balance, lycopodium, Silicea for inflammation and infection, thuja for breast wellness, as claimed in claim 1, and

12. This is a method of treating breast cancer with a formula and a means of delivery of the formula with a topical, topical, ductal, and/or interductal infusion. This is a formula and method of its delivery to abnormal breast tissue or to prevent the formation of cancer or precursors to cancer or atypia or other form of abnormal breast tissue, as claimed in claim 1, and

13. This formula and means to treat breast diseases all kinds may include homeopathic remedies that may be used topically or inside the breast and nipple with a method of delivery to those regions. This formula consists of a liquid anti disease anti cancer formula that can be delivered to the source where breast cancer begins, which is the ductal, topical and/or interductal system of the breast, with an topical infusion or via a suction and pumping action of the device to irrigate the breast tissue, or a topic means of delivery with the anti breast cancer anti breast inflammation anti breast cell atypia and hyperplasia and breast wellness formula as claimed in claim 1, and

14. This formula may use chemotherapy agents, anti-inflammatory agents, with or without ionic delivery system(s) for more reliable absorption into the breast with a topical agent or through the use of a device that is topical infusion or via a suction and pumping action of the device to irrigate the breast tissue with the anti breast cancer anti breast inflammation anti breast cell atypia and hyperplasia and breast wellness, as claimed in claim 1, and

15. This formula may include photonically programmable water and neutraceuticals in a form that has medicinal activities that may include but not limited to: decrease in oxidative stress to cells, support of healthy DNA, RNA, mitochondria with its anti-inflammatory, anti cancer, anti angiogenesis, and anti-aggregatory actions of stopping clots, by use of an alkaline charge of the water and minerals, preventing a buildup of elements responsible cancer or the precursors of cancer, or atypia or inflammatory tissue or cancerous cells, as claimed in claim 1, and

16. Fucoidan that can be used in this formula has many benefits that fight cancer. This includes block aptosis anti angiogensis, and other factors known and not known, and yet to be discovered. This an other nutrients may be used to block the adverse effect on synthesis of prostaglandins which are chemicals that play a part in inflammation and harmful thrombotic activities, acidosis, and adverse environmental terrain of the breast and surrounding tissue, and thereby becoming a breast wellness formula by treating or prevention of mutations of breast cells and tissues, and remediation of carcinogens, and restoring the homeostasis of the cells and tissues of the breast as claimed in claim 1, and

17. This formula may use of antioxidants with and without fucoidan, minerals that create an environment that are harmful to cancer and its precursors to cancer, or causing agents and other antioxidants that have anti-oxidant, anti-inflammatory, pH stabilizing, pain relieving effects and Nutraceutical benefits, beneficial effects against the aging process that is caused in part by inflammatory cells, damaged cells by oxidative stress, or degeneration of cells and/or the surrounding tissues as claimed in claim 1, and

18. This formula may include a Delivery System with or without trace minerals, antioxidants and nutrients that collectively decrease oxidative stress to cell with anti-inflammatory, and anti-aggregatory actions of stopping clots and build up of elements responsible for heart attack, stroke, and sudden cardiac arrest, blocks the adverse effect on synthesis of prostaglandins which are chemicals that play a part in inflammation and harmful thrombotic activities, the stable form and the selection of antioxidants in this formula inhibit inflammation that has been linked to chemicals or an acid state to the tissue or the accumulation of toxins in the tissue and with or without the use of nanoneutraceuticals. These nanoneutraceuticals are formed by use of this device wherein a nutrient can be made more useful for the body such as a delivery system for the nutrients as part of metabolic pathways and entrance into cells as claimed in claim 1, and

19. The photonic and ultraviolet processing of the formula may produce nano-sized nutraceutical particulate materials for greater bioavailability and usefulness for a breast wellness and anti breast disease formula. These nutrients are also photoceutical, with photonic energy processing the water or other nutrient for medicinal use with or with out the use of breast nipple catheter, breast ductal infusion and irrigation with a pump or use of a hand held device as claimed in claim 1, and

20. This is a means of breast infusion of the formula that may be done with breast ductal medicated infusion and irrigation with a pump or use of a hand held device or mechanical means of flow warm water around the nipple area of the breast and allow for flow into the nipple and inside of the breast ductal system and breast tissue, with or without a cup that allows for such a flow, with or without the assistance of a breast nipple topical and/or interductal catheter. There can also be pretreatment with a dilating agent for the nipple to allow for this medicated formula to enter the breast. This may include a topical nitroglycerin or other acceptable natural or pharmaceutical vasodilator for this purpose as claimed in claim 1, and

21. This is a medicated formula for breast diseases of all kinds or for prevention of breast diseases of all kinds with or without the use of liposomes for enhanced use of the formula included in this patent, with or without a device to assist delivery to the region of the breast and breast ductal region via the breast nipple as claimed in claim 1, and

22. Medicated formula with or without the use of liposomes, with or without the following: alkaline salts and other nutrients, anti-inflammatory agents, fucoidan, with or with one or more of the following chemo therapy agents such as but not limited to: Abraxane (paclitaxel protein-bound particles for injectable suspension), Adriamycin (doxorubicin), Aredia (generic name, pamidronate disodium), Arimidex (anastrozole), Aromasin (exemestane), Chemotherapy Regimens of different kinds known and not known and yet to be discovered, Cytoxan (cyclophosphamide), Ellence (epirubicin), Evista (raloxifene), Fareston (toremifene), Femara (letrozole), Herceptin (trastuzumab), Megace (megestrol), Tamoxifen, Taxol (paclitaxel), Taxotere (docetaxel), Xeloda (capecitabine), Zoladex (goserelin acetate), Zometa (generic name, zoledronic acid), and other agents as claimed in claim 1, and

23. The elements contained in this formula can be used for the treatment or prevention of breast diseases of all kinds, may be processed with or without photonic programming or an ionic delivery system for greater effectiveness and use against degenerative diseases or cancerous or precancerous diseases, precursors of breast disease, inflammatory breast disease, or accumulation of toxins in the breast tissue that tend to concentrate in the breast such as, but not limited to mercury, cadmium, lead, heavy metals, benzene, furans, organophosphates, petroleum derivates, and other toxic substances as claimed in claim 1, and

24. The elements contained in this formula for the treatment or prevention of breast diseases of all kinds may contain anti-inflammatory and reparative properties and as an anti-aging agent and wellness formula, with or without fucoidan for its benefits, and with or without alkaline minerals for homeostasis of breast tissue, with or without homeopathic formula for breast disease, hormonal balancing, and other nutrients as claimed in claim 1, and

25. Some examples of nutrients that can be used include: Omega Fish Oil Concentrate, Flax Seed Oil, Borage Oil. In addition, there may be a Mineral Bio-Matrix, Trace Minerals, MSM, Piper Nigrum as an ionic delivery system, and a Natural Antioxidant Complex that may consist of Rosemary Oil, Alpha Tocopherol, Astaxanthin, and Algal Oil. Algal Oil can be used as a stabilizing agent to prevent oxidation of the essential fatty acids that cause oxidative stress to cells when not stabilized as claimed in claim 1, and

26. Degenerative diseases such as breast cancer have been linked to a lack of nutrients as well as metabolic deficiencies, acid states, and chemical imbalances, toxins in food, acidosis, and toxins from the environment. The combination and individual ingredients in this formula have particular benefits on breast disease, cancerous and precancerous breast lesions, and breast degenerative disease as claimed in claim 1, and

27. Components in this formula have the abilities to assist the cells of the breast against inflammation and disease by its assisting with assimilation of toxins, removing, eliminating, diminishing, processing, scavaging free radials associated with cycles of cell disease, problems with cell and systemic metabolism, cellular acidosis, and the inflammatory process that causes atypia, hyperplasia, ductal carcinoma in situ, and breast disease that may be treated with this formula and method of delivery to the breast region, as claimed in claim 1, and

28. This formula has anti-inflammatory agents, plus a diffusion technology, and may have ionic delivery system(s) for more reliable absorption through the use of a form that is topical infusion or via a suction and pumping action of the device to irrigate the breast tissue with the anti breast cancer anti breast inflammation anti breast cell atypia and hyperplasia as claimed in claim 1, and

29. The nutrients may be selected from the following sources due to their quality, bioavailability, use, and concentration of essential fatty acids and the ability to be used with the stabilization process and with the ionic delivery process with this method of making an antioxidant health care product: Omega Fish Oil Concentrate, Flax Seed Oil, Borage Oil. In addition, there may be Ionic trace Minerals, MSM, Piper Nigrum as an ionic delivery system, and a Natural Antioxidant Complex that consists of Rosemary Oil, Alpha Tocopherol, Astaxanthin, and Algal Oil. Algal Oil may be used as a stabilizing agent to prevent oxidation of the essential fatty acids. These would be from organic sources as claimed in claim 1, and

30. These remedies can be given topically or infused into the breast or other mode of delivery in a homeopathic form or other acceptable form and may be selected for treating various conditions of the breast, especially breast cancer: Bufo vulgaris, Conium maculatum, Graphites, Mercurius, and Silicea. This is a formula that can used topically around the breast nipple or via infusion and may include the use of these and other homeopathic ingredients can be selected based on history of exposure to toxins and with clinical findings and subjective complaints, as well as the condition of the patient. These are formed from heavily diluted preparations; these may be used with effects similar to the disease's symptoms. This dilution is usually quite extensive, and often continues until no molecules of the original substance are likely to be found by routine testing. There are other homeopathic ingredients that can be included for their properties, such as but not limited to: apis can be used when there is inflammation, atypia, or hyperplasia of breast tissue. Arsenicum can be used if there is lowered immunity or breast cancer, or if nipples are retracted. Asterias can be used with swelling of breasts or with any stage of breast cancer, even in ulcerative stages. Baric iodide can be used with cancer of breasts, suspected autoimmune problem that is linked to the breast cancer or need for iodide. Belladonna, or Deadly Nightshade for breast inflammation and cancer. Bromium for swelling of breast tissue. Calcareas for precancerous conditions and local pain. Cancer nosode or carcinosinum for breast cancer. Chimaphila or pipsissewa for breast tumors. Conium for breast cancer and inflammation. Hepar sulphuris calcareum for breast cancer and for remediation of heavy and other metals. Cundurango for breast wellness, Hydrastis for immune support for breast cancer and precancerous conditions, Nitric acid for breast wellness, Phosphorus for cancer of breast, sorinum for breast inflammation, kalis for balance, lycopodium, Silicea for inflammation and infection, and/or thuja for breast wellness as claimed in claim 1, and

31. This is a method of treating and preventing breast cancer with a formula and a means of delivery of the formula with nipple, topical, ductal, and/or interductal penetration that can also be used as a patch, as claimed in claim 1, and

32. This formula and method of its delivery to abnormal breast tissue or to prevent the formation of cancer or precursors to cancer or atypia or other form of abnormal breast tissue, as claimed in claim 1, and

33. This formula consists of a liquid anti-disease, anti-cancer formula that can be delivered to the source where breast cancer begins, which is the ductal and topical system of the breast, and can be used topically with a nipple patch to allow for absorption inside the breast with repetitive use as claimed in claim 1, and

34. This is an topical and/or interductal infusion, irrigation or other mode of therapy or via a suction and pumping action of the device to irrigate the breast tissue with the anti breast cancer anti breast inflammation, anti breast cell atypia and hyperplasia, with or without use of a fucoidan for its Anti-aging and wellness formula in an ionic delivery system using alkaline minerals in an oxide state, trace minerals, herbal extracts, antioxidants and metabolic nutrients for cell health and the treatment of diabetes, heart disease, immune diseases, and may include the use of a soluble form fucoidan for its Anti-aging and wellness formula in an ionic delivery system using alkaline minerals in an oxide state, trace minerals, herbal extracts, antioxidants and metabolic nutrients for cell health and the treatment of diabetes, heart disease, immune diseases, as claimed in claim 1, and

35. This formula has anti-inflammatory agents, plus ionic delivery systems for more reliable absorption through the use of a form that is topical infusion or via a suction and pumping action of the device to irrigate the breast tissue with the anti breast cancer anti breast inflammation anti breast cell atypia and hyperplasia and breast wellness. This formula may be used in other regions of the body by various means of transport, delivery, infusion, injection, topical application or other means of use and delivery, and the formula may be different for the topical cream versus the injectable form, for example the injectable form may not contain a liposomal or other delivery system as the topical, and may not have certain oils or anti-inflammatory agents, and the preferred embodiment of the injectable form that can be via a needle, intravenous, topical or other suitable method of use and may only use a water soluble fucoidan solution with or without any of the following or any combinations of the above substances; alkaline salts or alkaline producing or supporting minerals such as but not limited to calcium, magnesium and potassium in an acceptable-form such as calcium gluconate, calcium chloride, calcium carbomate, potassium chloride, potassium gluconate, etc. and or a programmable water, medicated water, homeopathic water, water of all kinds, water treated or enhanced my machines and devices of all kinds, derivates of water such as hydrogen water, water with certain oscillatory or vibrational frequencies, or solutions that have been enhanced or the above formulation that can be enhanced by energy fields or some form of enhancement, and may include one or more of the following: Seaweed complex (nori, kombu, wakame, arame, dulsi, limu, other from Pac Northwest), Green tea extract, Tea complex (sencha, gyokuro, matcha, ginger root), Pomegranate extract, Berry complex (ellagic acid, polyphenols, glucaric acid (cherry)), Rice extract, Omega 3 and 6, Mushroom complex (shiitake, maitake, enoki, crimini, portobello, oyster, thistle oyster, turkey tail (polysaccarides, lentinian), Tumeric, Cinnamon, Apigine (parsely, celery), Curry, Marjoram, Oregano, Basil, Resveratrol, ALA (alpha-lipoic acid), Leeks, shallots, Chives, Vitamin D, Alkaline Complex, Trace Mineral Complex, Germanium Sesquioxide, MSM, as claimed in claim 1.
